(19) United States
(12) Patent Application Publication
(10) Pub. No.: US 2023/0035057 A1
(43) Pub. Date: Feb. 2, 2023

(54) METHOD OF TREATMENT OF SLEEP APNEA

(71) Applicant: PRIMAVERA BIOSCIENCES, INC., Columbia, MD (US)

(72) Inventor: Yoshinori Takeda, Columbia, MD (US)

(73) Assignee: PRIMAVERA BIOSCIENCES, INC., Columbia, MD (US)

(21) Appl. No.: 17/875,627

(22) Filed: Jul. 28, 2022

Related U.S. Application Data
(60) Provisional application No. 63/227,528, filed on Jul. 30, 2021.

(51) Int. Cl. A61K 31/137; A61K 31/197; A61K 31/4045; A61K 31/405; A61K 31/198; A61K 36/068; A61K 36/07; A61K 36/074; A61K 36/28; A61K 36/53; A61K 36/81; A61K 9/20

(52) U.S. Cl. CPC A61K 31/137; A61K 31/197; A61K 31/4045; A61K 31/405; A61K 31/198; A61K 36/068; A61K 36/07; A61K 36/074; A61K 36/28; A61K 36/53; A61K 36/81; A61K 9/20

(57) ABSTRACT

A method of treating sleep apnea is provided. The method including the step of administering 12.5 mg to 25.0 mg of ephedrine before going to bed to a patient in need of treating the sleep apnea. Preferably, 5.0 mg to 100.0 mg of the ephedrine is administered about one hour before going to bed, ephedra containing 5.0 mg to 100.0 mg of the ephedrine is administered, the ephedrine is administered 15 to 45 minutes before going to bed, and/or the ephedrine or ephedra containing the ephedrine is in a form of immediate release, extended release, or sustained release.

### CROSS-REFERENCE TO RELATED APPLICATIONS

This is a non-provisional application which claims priority of U.S. provisional application No. 63/227,528 filed on Jul. 30, 2021, the contents of which are incorporated by reference in its entirety.

### FIELD OF THE INVENTION

The present invention relates to a method of treating sleep apnea, more specifically, the use of ephedra or ephedrine to reduce breathing problems caused by obstructive sleep apnea.

### BACKGROUND OF THE INVENTION

Sleep apnea is a serious disease. One in five American adults, approximately 40 million people, suffer from obstructive breathing during sleep. More than 60% of people over the age of 65 snore during sleep and suffer from sleep deprivation.

Sleep apnea is characterized by instances of shallow breathing and/or frequent pauses in breathing caused by the airway collapsing during sleep. Obstructed breathing and gasping for air raise blood pressure as much as lifting heavy weights. When this happens all night, every night, year after year, it will certainly shorten one's life.

Sleep apnea causes high blood pressure, cardiovascular disease, stroke, Alzheimer's disease, dementia, and obesity.

Drowsiness and fatigue resulting from sleep apnea causes impaired performance at work and motor vehicle and heavy machine-related accidents.

Ever since W. C. Dement, MD, PhD (Stanford U) advocated for the seriousness of this disease, sleep centers have been established throughout the country.

Once diagnosed, however, remedy options are still limited.
An FDA-approved device, called the CPAP (Continuous Positive Airway Pressure) device, circumvents this problem. However, since CPAP devices are cumbersome, many sleep apnea patients stop using them.
Oral appliances (mouth guards) or surgical modifications of the airway have limited success.
The FDA approved an implant device “Inspire” that electrically stimulates the pharynx muscles to keep them from relaxing. A limited number of people have taken this expensive procedure ($20,000-$50,000) but it is not clear that the device works in real-life practice.
People are encouraged to sleep on their side to reduce airway blockage. However, this often fails.

Although there are numerous drugs that deal with various sleep problems, none directly treats the breathing problems caused by sleep apnea. Although there are numerous sleeping pills (supplements), none of them helps reduce the breathing problems caused by sleep apnea. Such is our current understanding (knowledge) of sleep apnea problems. There is a need of a new, noteworthy treatment for sleep apnea, there is no report for such treatment.

### SUMMARY OF THE INVENTION

Extensive research has been made on herbs that affect breathing directly or indirectly and tested several of them to see if any herbs help with sleep apnea. It has been found that ephedra or ephedrine reduces the breathing problems caused by sleep apnea.

In accordance with embodiments of the present invention, a method of treating sleep apnea includes the step of administering of 5.0 mg to 100.0 mg of ephedrine before going to bed to a patient in need of treating the sleep apnea. In one embodiment, 12.5 mg to 25.0 mg of the ephedrine is administered about one hour before going to bed, ephedra containing 5.0 mg to 100.0 mg of the ephedrine is administered, the ephedrine is administered 15 to 45 minutes before going to bed, and/or the ephedrine or ephedra containing the ephedrine is in a form of immediate release, extended release, or sustained release. In another embodiment, a sleeping pill containing the ephedrine is administered. In yet another embodiment, the ephedrine is administered in combination with other ingredients. For example, the sleeping pill may further contain at least one ingredient selected from the group consisting of chamomile, hops, passionflower, lemon balm, valerian, melatonin, GABA, L-theanine, L-tryptophan, 5-HTP, ashwagandha, schizandra, reishi, maitake, Ganoderma lucidum, lion's mane, chaga, or Cordyceps, and any combination thereof.

In another aspect, a sleep apnea treatment kit including the sleeping pill is provided. The kit includes at least the sleeping pill containing 5.0 mg to 100.0 mg or more preferably 12.5 mg to 25.0 mg of ephedrine, and instructions for a patient to take the sleeping pill before going to bed in need of treating the sleep apnea. In one embodiment, the sleeping pill contains the ephedrine in a form of immediate release, extended release, or sustained release thereof. The instructions may include instructions for the patient to take the sleeping pill 15 to 45 minutes before going to bed.

### BRIEF DESCRIPTION OF THE DRAWINGS

FIG. 1 is a diagram showing sleep apnea patient's oxygen saturation levels (SpO2). Oxygen saturation levels (SpO2) in the blood during sleep were monitored using Wellure's O2Ring.

FIG. 2 is a diagram showing sleep apnea patient's oxygen saturation levels (SpO2) when a patient took 25 mg of ephedrine one hour before going to bed along with a sleeping pill Avinol PM just before going to bed. Oxygen saturation levels (SpO2) in the blood during sleep were monitored using Wellure's O2Ring.

FIG. 3 is a diagram showing sleep apnea patient's oxygen saturation levels (SpO2) when a patient took 25 mg of ephedrine one hour before going to bed along with one tablet of the sleeping pill Avinol PM just before going to bed. The patient took another 25 mg of ephedrine three hours after going to bed. Oxygen saturation levels (SpO2) in the blood during sleep were monitored using Wellure's O2Ring.

### DETAILED DESCRIPTION OF EMBODIMENTS

Ephedra (ma huang) has been used as a respiratory bronchodilator in Chinese medicine since ancient times (>2,000 years). Ephedrine is the main active ingredient of ephedra. In 1885, Japanese organic chemist Nagai Nagayoshi chemically synthesized ephedrine, and its industrial manufacturing in China began in the 1920s, when Merck began marketing and selling the drug as ephetonin.

Ephedrine belongs to a class of compounds called a catecholamine (neurotransmitter) and is chemically and structurally related to norepinephrine, a sympathetic nervous system (SNS) stimulant similar to amphetamines. Ephedrine interacts with the adrenergic receptors (alpha and beta receptors), increases the activity of norepinephrine, and helps release noradrenaline and dopamine in the substantia nigra. Since the adrenergic receptors are prevalent and found not only in the SNS (brain) but also in many cells throughout the body, ephedra or ephedrine affects many cells throughout the body.

Ephedrine is a potentially dangerous natural compound. As of 2004 the US Food and Drug Administration (FDA) had received over 18,000 reports of adverse effects in people using it. Cardiovascular problems, hypertension, flushing, sweating, nausea, difficulty urinating, restlessness, confusion, insomnia, hallucination, agitation, and many other problems are reported. Because of this, in 2004, the FDA created a ban on ephedrine alkaloids marketed for reasons other than asthma, colds, allergies, other diseases, or traditional Asian use. Subsequently, ephedrine was banned by the NCAA, MLB, NFL, and PGA. Ephedrine is, however, still legal in many applications outside of dietary supplements. Purchasing is currently limited and monitored, with specifics varying from state to state. George W. Bush signed the Combat Methamphetamine Epidemic Act of 2005. The federal statute includes these requirements for merchants who sell ephedrine:
A retrievable record of all purchases identifying the name and address of each party to be kept for two years
Required verification of proof of identity of all purchasers
Required protection and disclosure methods in the collection of personal information

Legality of ephedrine use (and purchase) varies from country to country. Canada, Australia, South Africa, Germany, and Japan have restrictions, but Japan has eased restrictions this year.

It has been found that a sleeping pill or a kit including the pill that contains a therapeutically effective amount of ephedrine can significantly improve apnea patient's oxygen saturation levels (SpO2), and thus is useful in effectively treating the sleep apnea.

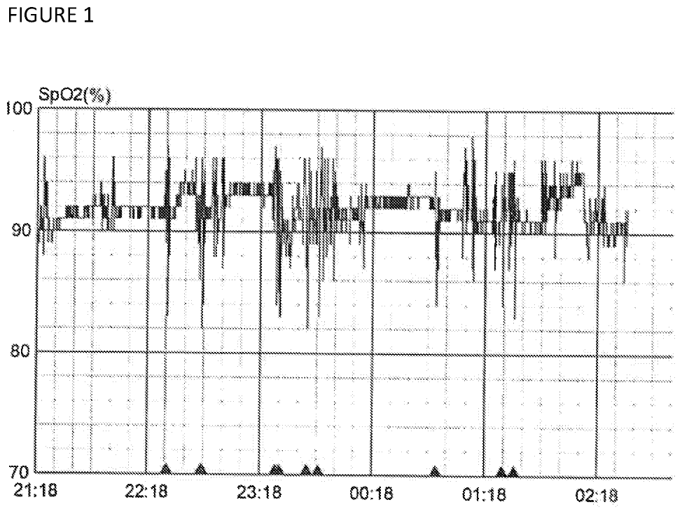

As shown in FIG. 1, sleep apnea patient's oxygen saturation levels (SpO2) were wildly fluctuated during sleep. A few breathing interruptions like these are enough to ruin a good night's sleep.

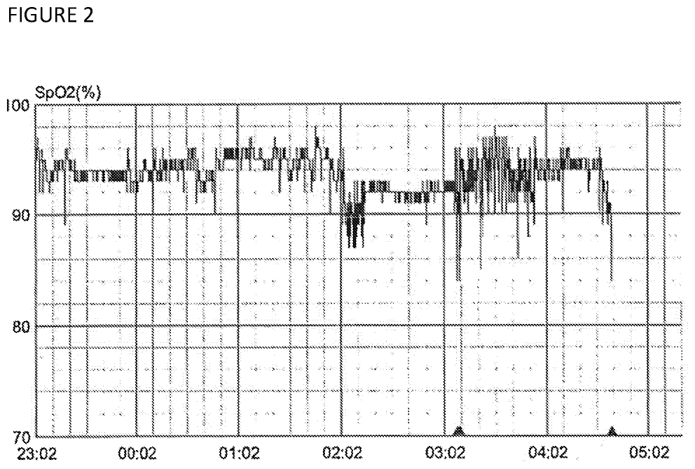

As shown in FIG. 2, the sleep apnea patient's oxygen saturation levels (SpO2) were improved when the patient took 25 mg of ephedrine one hour before going to bed along with a sleeping pill Avinol PM (trademark) just before going to bed. Breathing problems observed in FIG. 1 were greatly reduced in the first three hours but came back when the ephedrine was metabolized in about four hours.

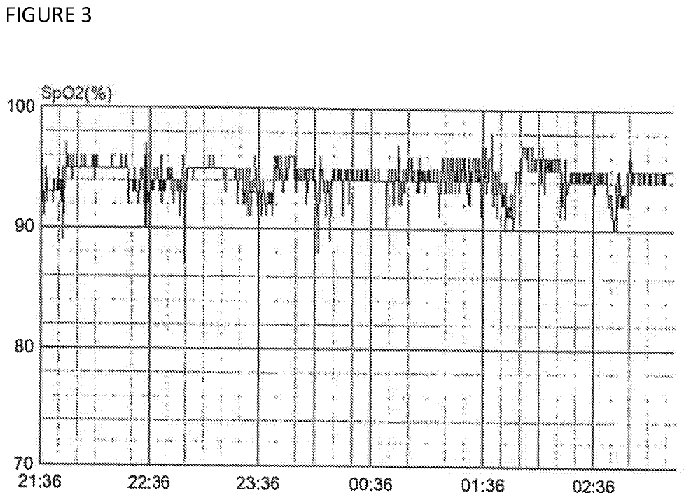

As shown in FIG. 3, the sleep apnea patient's oxygen saturation levels (SpO2) were improved when the patient took 25 mg of ephedrine one hour before going to bed along with one tablet of the sleeping pill Avinol PM (trademark) just before going to bed. The patient took another 25 mg of ephedrine three hours after going to bed.

FIGS. 2 and 3 indicate that ephedrine can preferably be taken in the sustained or extended release form to keep it active throughout the night.

The ephedrine may be administered in combination with other ingredients or medications. For example, at least one ingredient selected from chamomile, hops, passionflower, lemon balm, valerian, melatonin, GABA, L-theanine, L-tryptophan, 5-HTP, ashwagandha, schizandra, reishi, maitake, Ganoderma lucidum, lion's mane, chaga, or Cordyceps, and any combination thereof may be administered in appropriate combinations with ephedrine and amounts to counter (reduce) stimulatory (agitating) effects of the ephedra or ephedrine on sleep.

The sleep apnea treatment with ephedrine may be provided in the form of a kit. The kit may include the sleeping pill that contains a therapeutically effective amount of ephedrine and appropriate instructions for a patient who needs sleep apnea treatment to take the pill in the manner disclosed in the present application.

In another aspect, a pharmaceutical composition for treating sleep apnea is provided which includes a therapeutically effective amount of ephedrine, for example, 5.0 mg to 100.0 mg, or preferably 12.5 mg to 25.0 mg of the ephedrine, and optionally any pharmaceutically acceptable carrier, and optimally any other ingredients disclosed in the present application. In another aspect, use of the ephedrine for the manufacture of the pharmaceutical composition for treating sleep apnea is provided.

What is claimed is:

1. A method of treating sleep apnea, comprising administering 5.0 mg to 100.0 mg of ephedrine before going to bed to a patient in need of treating the sleep apnea.

2. The method according to claim 1, wherein 12.5 mg to 25.0 mg of the ephedrine is administered about one hour before going to bed.

3. The method according to claim 1, wherein ephedra containing 5.0 mg to 100.0 mg of the ephedrine is administered.

4. The method according to claim 1, wherein the ephedrine is administered 15 to 45 minutes before going to bed.

5. The method according to claim 1, wherein the ephedrine or ephedra containing the ephedrine is in a form of immediate release, extended release, or sustained release.

6. The method according to claim 1, wherein a sleeping pill containing the ephedrine is administered.

7. The method according to claim 6, wherein the sleeping pill further contains an ingredient selected from the group consisting of chamomile, hops, passionflower, lemon balm, valerian, melatonin, GABA, L-theanine, L-tryptophan, 5-HTP, ashwagandha, schizandra, reishi, maitake, Ganoderma lucidum, lion's mane, chaga, or Cordyceps, and any combination thereof.

8. A sleep apnea treatment kit comprising a sleeping pill, the kit comprising:
the sleeping pill containing 5.0 mg to 100.0 mg of ephedrine; and
instructions for a patient to take the sleeping pill before going to bed in need of treating the sleep apnea.

9. The kit according to claim 8, wherein the sleeping pill contains the ephedrine in a form of immediate release, extended release, or sustained release thereof.

10. The kit according to claim 8, wherein the instructions comprise instructions that instruct the patient to take the sleeping pill 15 to 45 minutes before going to bed.
